(19) United States
(12) Patent Application Publication
(10) Pub. No.: US 2008/0119867 A1
(43) Pub. Date: May 22, 2008

(54) PUNCTURE AND ABRASION RESISTANT SHEATH

(75) Inventor: Kevin L. Delaney, Bloomington, IN (US)

Correspondence Address: BRINKS HOFER GILSON & LIONE/CHICAGO/COOK, PO BOX 10395, CHICAGO, IL, 60610, US

(73) Assignee: COOK INCORPORATED, BLOOMINGTON, IN (US)

(21) Appl. No.: 11/927,954

(22) Filed: Oct. 30, 2007

Related U.S. Application Data
(60) Provisional application No. 60/855,497, filed on Oct. 31, 2006.

(51) Int. Cl. A61M 25/01

(52) U.S. Cl. 606/108; 604/500

(57) ABSTRACT

A delivery apparatus for introducing an implant, such as a vena cava filter, for capturing emboli in a body vessel. The apparatus includes an outer sheath having a tubular wall defining a lumen formed therethrough and having a proximal end extending to a distal end. The tubular wall includes an inner surface and may also include optional reinforcing members. A radiopaque marker band is disposed about the inner surface adjacent the distal end, and a tubular liner is disposed along the inner surface. The tubular liner comprises at least one of polyimide, PEEK, and PVDF. An inner catheter is slidably disposed within the lumen of the outer sheath and has a distal portion configured to engage and deliver the implant through the distal end of the outer sheath in the body vessel.

### CROSS-REFERENCE TO RELATED APPLICATIONS

This application claims the benefit of U.S. Provisional Application Ser. No. 60/855,497 filed on Oct. 31, 2006, entitled “PUNCTURE AND ABRASION RESISTANT SHEATH,” the entire contents of which are incorporated herein by reference.

### BACKGROUND

1. Field of the Invention

The present invention generally relates to delivery apparatus for vascular implants. More specifically, the invention relates to an introduction sheath having a protective liner for use with a vena cava filter.

2. Description of Related Art

Delivery apparatus for vascular implants include, for example, catheters having outer sheaths. An outer sheath must be conducive to navigating the tortuous bends found in blood vessels of the vascular system while also protecting the blood vessels from damage by the implants. Since the outer sheath is usually made of a soft, flexible material there is a potential for damage to the outer sheath caused by stiff or sharp portions of the implants. As the catheter is advanced through a tight radius in the vasculature, the implant may contact an inner surface of the outer sheath. Consequently, any sharp portions, such as hooks, of the implant may puncture the outer sheath, prematurely contacting and possibly damaging the blood vessel. In addition, the sharp or stiff portions may also abrade the inner surface, potentially introducing particulate matter from the outer sheath into the blood vessel.

In view of the above, it is apparent that there exists a need for an improved delivery apparatus for introducing implants into the vasculature.

### SUMMARY

In satisfying the above need, as well as overcoming the enumerated drawbacks and other limitations of the related art, the present invention provides a delivery apparatus for introducing an implant for capturing emboli in a body vessel. The apparatus includes an outer sheath having a tubular wall defining a lumen formed therethrough and having a proximal end extending to a distal end. The tubular wall may include optional reinforcing members. The tubular wall has an inner surface and may include a radiopaque marker band disposed adjacent the distal end. A tubular liner is disposed along the inner surface for improved puncture and abrasion resistance. The tubular liner comprises at least one of polyimide, polyetheretherketones (PEEK), and polyvinylidene fluoride (PVDF).

An inner catheter is slidably disposed within the lumen of the outer sheath. The inner catheter has a proximal portion extending to a distal portion and longitudinally moves relative to the outer sheath. The inner catheter engages and delivers the implant through the distal end of the outer sheath in the body vessel.

In one embodiment, the implant includes a vena cava filter having a proximal segment extending to a distal segment including a retrieval hook. A plurality of diverging spring-biased centering legs and a plurality of spring-biased anchoring legs are attached together at the distal end of the filter. The centering legs provide a force to center the implant in the body vessel, and the anchoring legs have proximal free ends including hooks for engaging the body vessel. The hooks of the anchoring legs are oriented to face toward the tubular liner when engaging the distal portion of the inner catheter.

In some examples, the proximal free ends releasably engage the distal portion of the inner catheter. In others, the orientation of the implant is reversed and the retrieval hook releasably engages the distal portion of the inner catheter.

In some embodiments, the outer sheath is made of one of nylon, high density polyethylene, polytetrafluroethylene and fluorinated ethylene-propylene. In addition, the reinforcing members may be made of stainless steel, high strength plastic and mixtures thereof. The high strength plastic may include aramid fibers.

In another embodiment, a thin coating material is disposed between the inner surface and the tubular liner to bond the tubular liner to the outer sheath. The thin coating material includes nylon, copolyamide and urethane.

In yet another embodiment, a radiopaque marker band is included and is disposed between the inner surface and the tubular liner. The material of the radiopaque marker band is at least one of platinum, gold, silver, palladium, tungsten, niobium, cobalt, and copper.

A method of introducing an implant into a body vessel includes providing a delivery apparatus having an outer sheath comprising a tubular wall having an inner surface and defining a lumen formed therethrough. A tubular liner comprising at least one of polyimide, PEEK, and PVDF is disposed along the inner surface, a radiopaque marker band is disposed near the distal end of the outer sheath, and an inner catheter is slidably disposed within the lumen of the outer sheath.

The method also includes inserting the implant within the lumen such that a proximal free end of the implant is coupled to a distal portion of the inner catheter and the implant is adjacent a distal end of the delivery apparatus. The distal end of the apparatus is positioned adjacent a desired location in the body vessel, the outer sheath is retracted proximally from the implant, and the implant is released into the body vessel.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1a is an environmental view of a distal portion of a delivery apparatus according to the present invention introducing a vena cava filter into a body vessel;

FIG. 1b is a sectional view of a portion of the delivery apparatus of FIG. 1a showing a proximal free end of the vena cava filter engaging a distal portion of an inner catheter;

FIG. 1c is an environmental view of a distal portion of an alternate embodiment of the delivery apparatus of the present invention introducing the vena cava filter into the body vessel;

FIG. 1d is a sectional view of a portion of the delivery apparatus of FIG. 1c showing a retrieval hook of the vena cava filter engaging the distal portion of the inner catheter;

FIG. 2 is a sectional view of an outer sheath of the delivery apparatus of FIG. 1a or 1c;

FIG. 3a is a detail view of a distal portion of the outer sheath of FIG. 2;

FIG. 3b is a detail view of a segment of a wall of the outer sheath of FIG. 2;

FIG. 4a is a side view of a delivery assembly according to one embodiment of the present invention;

FIG. 4b is an exploded side view of the assembly of FIG. 4a; and

FIG. 5 is a flow chart describing a method of filtering emboli using the delivery apparatus and vena cava filter of FIG. 1a or 1c.

### DETAILED DESCRIPTION

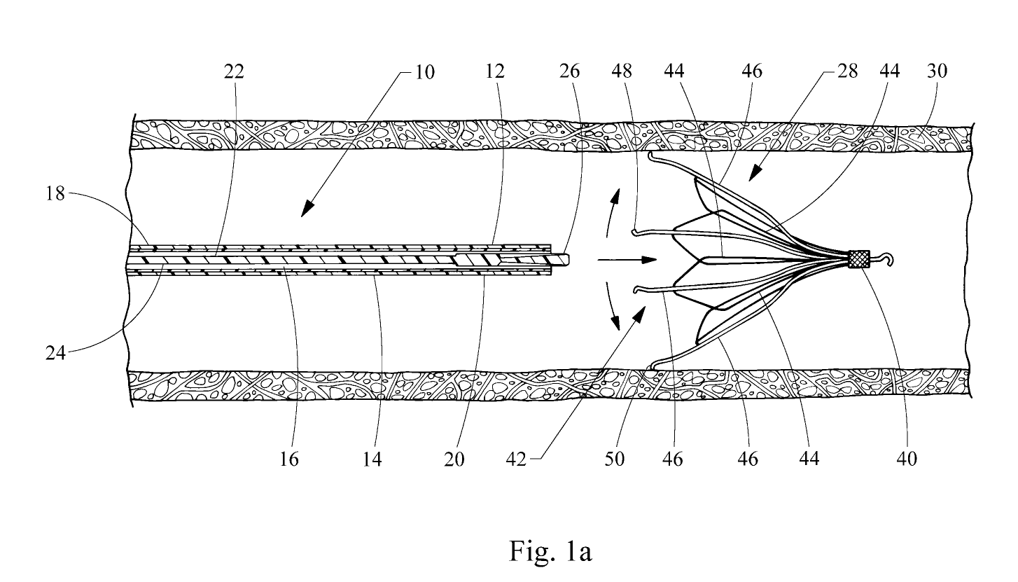

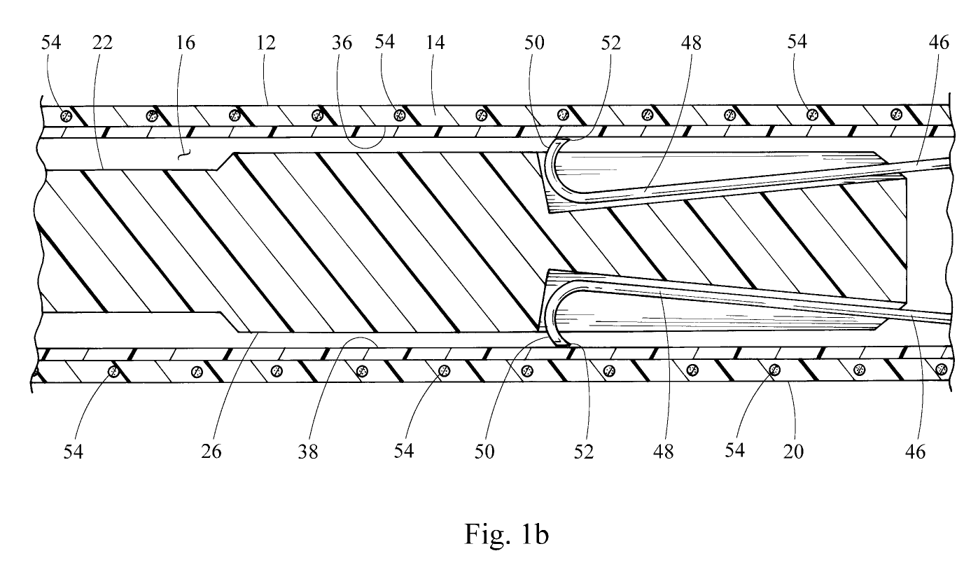

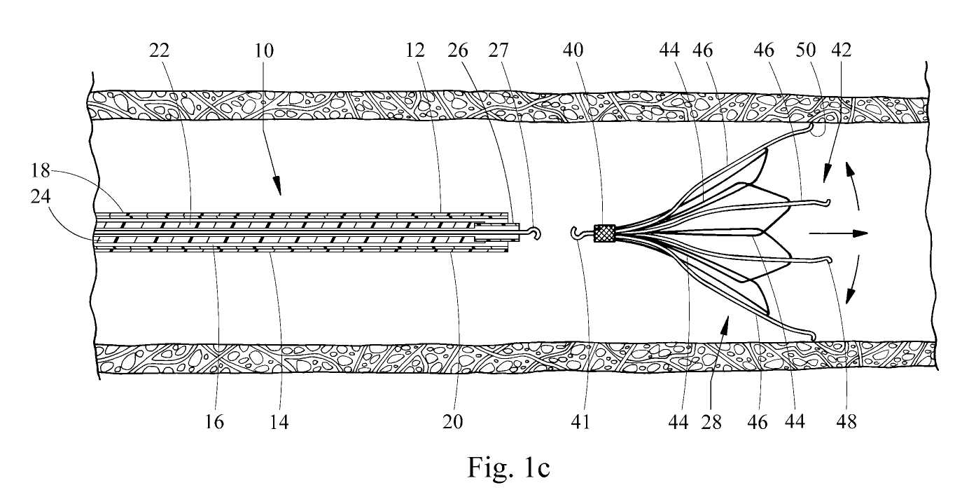

Referring now to FIG. 1a, a delivery apparatus embodying the principles of the present invention is illustrated therein and designated at 10. As its primary components, the apparatus 10 includes an outer sheath 12 having a proximal end 18 extending to a distal end 20 and a tubular wall 14 defining a lumen 16 formed therethrough. The apparatus 10 further includes an inner catheter 22 slidably disposed within the lumen 16 of the outer sheath 12. The inner catheter 22 has a proximal portion 24 extending to a distal portion 26 and is configured to longitudinally move relative to the outer sheath 12 and to engage and deliver an implant 28 through the distal end 20 of the outer sheath 12 into a body vessel 30.

FIG. 1a shows a vena cava filter, but the implant 28 may include any device appropriate for capturing emboli in the body vessel 30, or any other appropriate implant, such as a stent. The vena cava filter of FIG. 1a has a proximal segment 42 extending to distal segment 40. A plurality of spring-biased centering legs 44 and a plurality of spring-biased anchoring legs 46 are attached together at the distal segment 40 such that in an unrestrained state the legs 44, 46 diverge radially in a proximal direction from the distal segment 40. As a result, at least part of the centering legs 44 and the anchoring legs 46 may contact the body vessel 30 when the vena cava filter 28 is deployed.

When the centering legs 44 contact and are radially restrained by the body vessel 30, the centering legs 44 provide a centering force. The centering force acts to radially center the implant 28 within the body vessel 30. The anchoring legs 46 include proximal free ends 48 having, for example, anchoring hooks 50. The anchoring hooks 50 are configured to engage the body vessel 30 to axially secure the filter 28 within the body vessel 30.

As best shown in the example of FIG. 1b, when the implant 28 is restrained within the lumen 16 of the outer sheath 12, the proximal free ends 48 of the anchoring legs 46 are configured to releasably engage the distal portion 26 of the inner catheter 22. When, for example, the outer sheath 12 is retracted proximally relative to the inner catheter 22 the distal segment 40 emerges first before the spring-biased anchoring legs 46 release from the distal portion 26. Additionally, points 52 of the anchoring hooks 50 of this embodiment are oriented to face toward the outer sheath 12. Since it may be desirable to minimize a diameter of the outer sheath 12, the points 52 may be in close proximity to the outer sheath 12. As a result, there is a significant probability that the points 52 will contact, and possibly damage, the outer sheath 12 as the delivery apparatus 10 and the implant 28 is advanced through various bends of the body vessel 30.

In another embodiment of the present invention, shown in the example of FIGS. 1c and 1d, the orientation of the implant 28 is reversed from that of FIG. 1a. The distal segment 40 includes a retrieval hook 41 used for both retrieval and deployment of the implant 28. In addition, as best shown in FIG. 1d, the distal portion 26 of the inner catheter 22 further includes a deployment snare 27 attached to, for example, a proximally extending release wire 23. As a result, when the outer sheath 12 is retracted proximally relative to the inner catheter 22, the anchoring hooks 50 of the anchoring legs 46 emerge before the retrieval hook 41 is detached from the deployment snare 27. Since the anchoring hooks 50 do not engage the distal portion 26 of the inner catheter 22, there is an even greater probability that the points 52 of the anchoring hooks 50 will contact and damage the outer sheath 12 as the delivery apparatus 10 and the implant 28 are advanced through various bends of the body vessel 30.

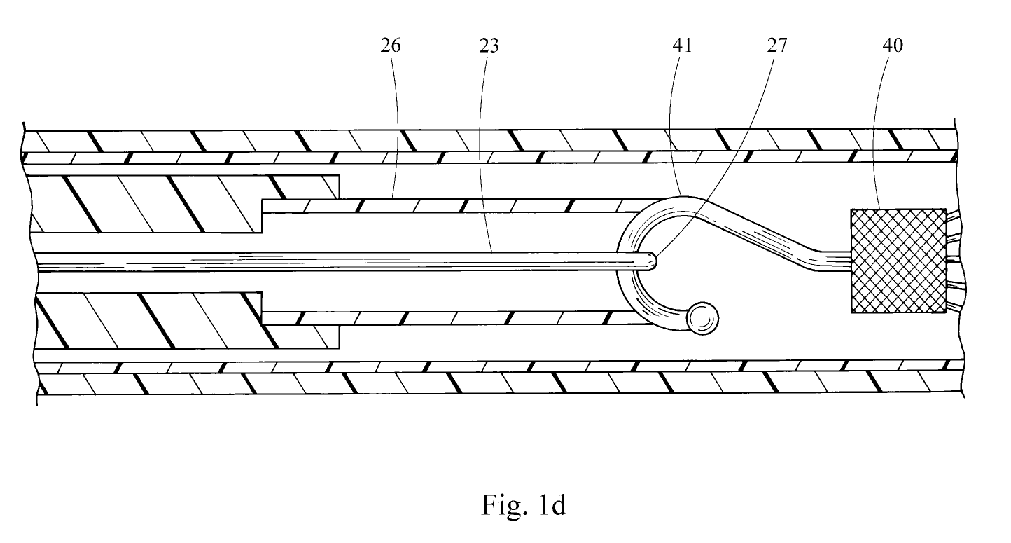

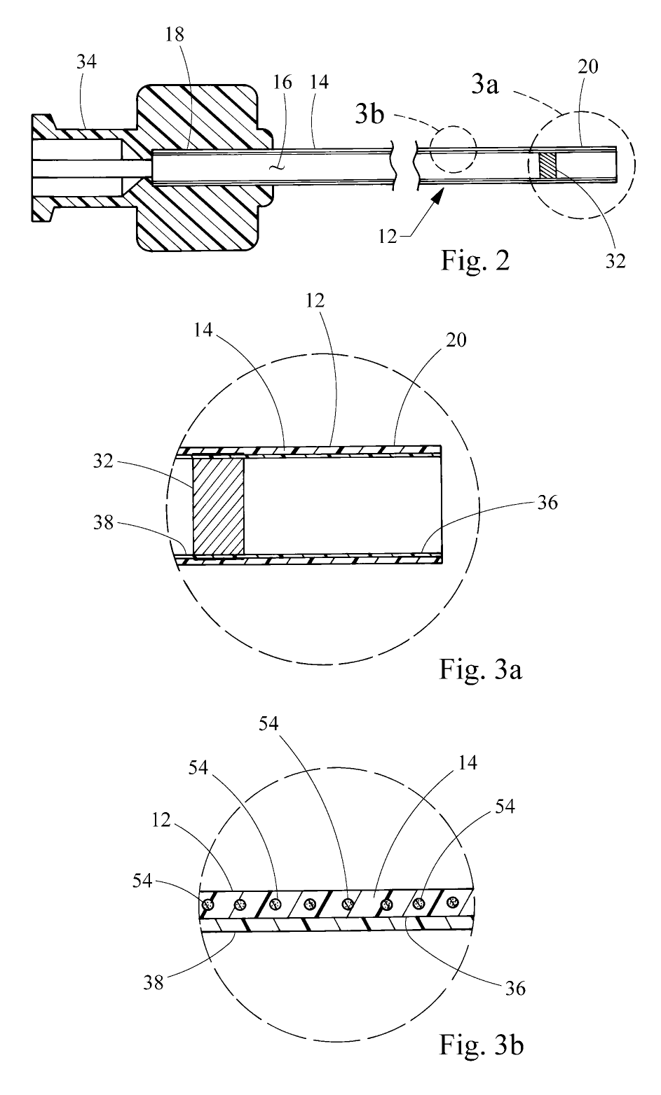

Turning to FIGS. 2 and 3, the outer sheath 12 is shown in more detail. The outer sheath 12 includes a radiopaque marker band 32 adjacent the distal end 20 and a hub 34 at the proximal end 18. In order to protect the outer sheath 12 from puncture and abrasion by, for example, the anchoring hooks 50 of the implant 28, a tubular liner 38 is disposed along the length of an inner surface 36 as best shown in FIGS. 3a and 3b. Preferably, the tubular liner 38 is made of polyimide (PI), polyetheretherketones (PEEK), or polyvinylidene (PVDF). Polyimide, PEEK, and PVDF are thermoplastics or thermoset polycondensates with high impact strength.

In yet another embodiment, the radiopaque marker band 32 is disposed on the inner surface 36, between the tubular liner 38 and the tubular wall 14. The radiopaque marker band 32 may be formed of a number of materials that are visible under, for example, fluoroscopy. These include, but are not limited to, platinum, gold, silver, palladium, tungsten, niobium, cobalt, and copper. The purpose of the marker band 32 is to allow a physician to determine the location of the proximal end 18 of the outer sheath 12 within the body vessel 30.

The hub 34 is configured to provide access to the lumen 16 of the outer sheath 12 to allow the inner catheter 22 to be disposed within the lumen 16. Thus, the physician is able to manipulate both the outer sheath 12 and the inner catheter 22 for relative longitudinal movement. Also, the entire delivery apparatus 10 may be moved proximally or distally within the body vessel 30.

The tubular wall 14 of the outer sheath 12 may be made of a number of materials including nylon, high density polyethylene (HDPE), polytetrafluroethylene (PTFE), and fluorinated ethylene-propylene (FEP). To provide enhanced fracture resistance, optional reinforcing members 54 may also be disposed within the tubular wall 14 as shown in FIG. 3b. The reinforcing members 54 may be made of stainless steel, high strength plastic and mixtures thereof. The high strength plastic may include aramid fibers. Aramid fibers are fibers formed from a long-chain polyamide and may include para-aramid fibers. The reinforcing members 54 may be braided reinforcing members, coils, or any other suitable type of reinforcing members. Reinforcing members 54 in the form of coils could be as disclosed in U.S. Pat. No. 5,700,253, which is hereby incorporated by reference in its entirety.

In some embodiments, a thin coating material (not shown) may be disposed between the inner surface 36 and the tubular liner 38 to bond the tubular liner 38 to the inner surface 36. Examples of the thin coating material include nylon, copolyamide and urethane. These materials may, for example, be applied upon the inner surface 36, the tubular liner 38 or upon both features prior to disposing the tubular liner 38 within the tubular wall 14 of the outer sheath 12.

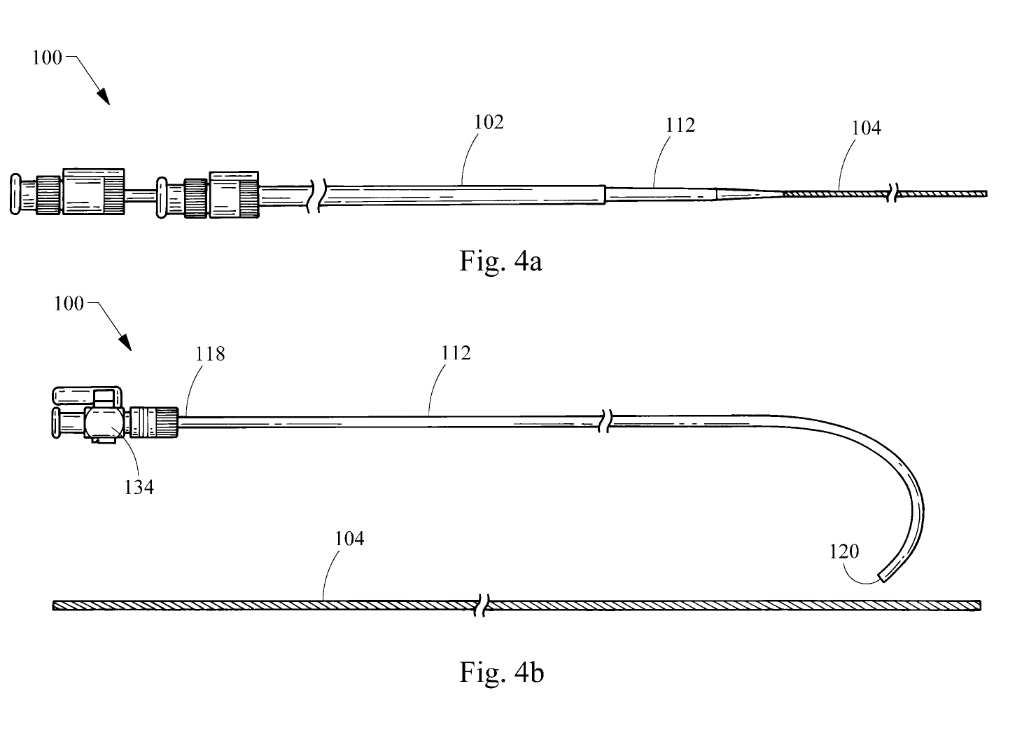

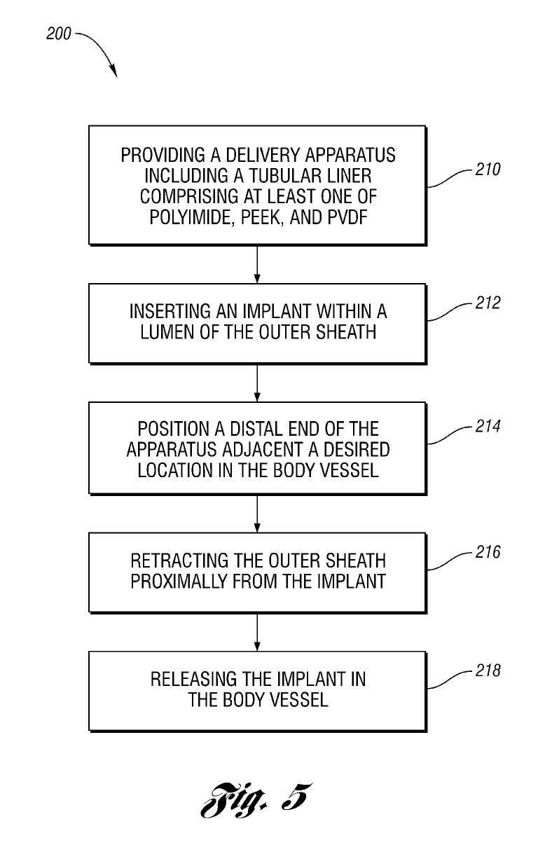

FIGS. 4a and 4b depict a delivery assembly 100 for introducing a vena cava filter for capturing emboli in a body vessel in accordance with another embodiment of the present invention. As shown, the delivery assembly 100 comprises the delivery apparatus 10 described above and further includes a vena cava filter (not shown). The delivery assembly 100 shares similar features with the delivery apparatus 10. Such features share the same reference number indexed by 100.

In this example, the assembly 100 further includes a polytetrafluoroethylene (PTFE) introducer sheath 102 for percutaneously introducing a wire guide 104 and an outer sheath 112 (equivalent to the outer sheath 12 above) in a body vessel. Of course, any other suitable material for the introducer sheath 102 may be used without falling beyond the scope or spirit of the present invention. The outer sheath 112 includes a tubular liner to provide puncture and abrasion resistance. The tubular liner comprises at least one of polyimide, PEEK, and PVDF. The introducer sheath 102 may have any suitable size, for example, between about three-french to eight-french. The introducer sheath 102 serves to allow the outer sheath 112 and an inner catheter to be percutaneously inserted to a desired location in the body vessel. The introducer sheath 102 receives the outer sheath 112 and provides stability to the outer sheath 112 at a desired location of the body. For example, the introducer sheath 102 is held stationary within a common visceral artery, and adds stability to the outer sheath 112, as the outer sheath 112 is advanced through the introducer sheath 102 to a desired area in the vasculature.

As shown, the assembly 100 may also include a wire guide 104 configured to be percutaneously inserted within the vasculature to guide the outer sheath 112 to the filtration area. The wire guide 104 provides the outer sheath 112 with a path to follow as it is advanced within the body vessel. The size of the wire guide 104 is based on the inside diameter of the outer sheath 112.

When a distal end 120 of the outer sheath 112 is at the desired location in the body vessel, the wire guide 104 is removed and the vena cava filter, having a distal retrieval hook releasably coupled to a distal portion of the inner catheter, is inserted into the outer sheath 112. The inner catheter is advanced through the outer sheath 112 for deployment of the vena cava filter through the distal end 120 to capture emboli in the body vessel.

As shown, the outer sheath 112 has a proximal end 118 and a hub 134 to receive the inner catheter and vena cava filter for advancement through the outer sheath 112. The size of the outer sheath 112 is based on the size of the body vessel in which it percutaneously inserts, and the size of the vena cava filter.

In this embodiment, the vena cava filter and inner catheter are coaxially disposed through the outer sheath 112, following removal of the wire guide 104, in order to position the vena cava filter in position to capture emboli in the body vessel. The vena cava filter is guided through the outer sheath 112 by the inner catheter, preferably from the hub 134, and exits from the distal end 120 of the outer sheath 112 at a location within the vasculature where filtration is desired.

It is understood that the assembly described above is merely one example of an assembly that may be used to deploy the vena cava filter, in conjunction with the outer sheath 112, in the body vessel. Of course, other apparatus, assemblies and systems may be used to deploy any embodiment of the embolic protection device without falling beyond the scope or spirit of the present invention.

FIG. 5 provides a flow chart designated at 200 describing a method for introducing an implant into a body vessel. The method 200 includes percutaneously introducing a delivery apparatus including an outer sheath in the body vessel in box 210. The outer sheath includes a tubular wall defining a lumen having an inner surface upon which a tubular liner comprising at least one of polyimide, PEEK, and PVDF is disposed. A radiopaque marker band is disposed near a distal end of the outer sheath and an inner catheter is slidably disposed within the lumen of the outer sheath. At box 212 the method 200 further includes inserting the implant within the lumen such that a proximal free end of the implant releasably engages a distal portion of the inner catheter. The method 200 also includes positioning the distal end of the apparatus adjacent a desired location in the body vessel at box 214, retracting the outer sheath proximally from the implant at box 216, and releasing the implant into the body vessel at box 218.

As a person skilled in the art will readily appreciate, the above description is meant as an illustration of implementation of the principles this invention. This description is not intended to limit the scope or application of this invention in that the invention is susceptible to modification, variation and change, without departing from the spirit of this invention, as defined in the following claims.

I claim:

1. A delivery apparatus for introducing an implant for capturing emboli in a body vessel, the apparatus comprising:
an outer sheath comprising a tubular wall defining a lumen formed therethrough and having a proximal end extending to a distal end, the tubular wall having an inner surface, a tubular liner disposed along the inner surface, the tubular liner comprising at least one of polyimide, polyetheretherketones (PEEK), and polyvinylidene fluoride (PVDF); and
an inner catheter slidably disposed within the lumen of the outer sheath, the inner catheter having a proximal portion extending to a distal portion, the inner catheter being configured to longitudinally move relative to the outer sheath to engage and deliver the implant through the distal end of the outer sheath in the body vessel.

2. The apparatus according to claim 1, wherein the implant includes a vena cava filter including a proximal segment extending to a distal segment, a plurality of diverging spring-biased centering legs and a plurality of diverging spring-biased anchoring legs being attached together at the distal segment of the filter, the centering legs providing a force to center the implant in the body vessel; each of the anchoring legs having a proximal free end including an anchoring hook for engaging the body vessel.

3. The apparatus according to claim 2, wherein the proximal free ends of the anchoring legs releasably engage the distal portion of the inner catheter.

4. The apparatus according to claim 3, wherein the hooks of the anchoring legs are oriented to face toward the tubular liner when engaging the distal portion of the inner catheter.

5. The apparatus according to claim 2, wherein the distal segment of the filter further includes a retrieval hook, the retrieval hook releasably engaging the distal portion of the inner catheter.

6. The apparatus according to claim 1, wherein the outer sheath is made of one of nylon, high density polyethylene, polytetrafluroethylene and fluorinated ethylene-propylene.

7. The apparatus according to claim 1, wherein reinforcing members are disposed within the tubular wall.

8. The apparatus according to claim 7, wherein the reinforcing members are made of stainless steel or high strength plastic or a mixture thereof.

9. The apparatus according to claim 8, wherein the high strength plastic includes aramid fibers.

10. The apparatus according to claim 1, wherein a thin coating material is disposed between the inner surface and the tubular liner to bond the tubular liner to the outer sheath.

11. The apparatus according to claim 10, wherein the thin coating material includes one of nylon, copolyamide and urethane.

12. The apparatus according to claim 1, wherein the outer sheath further comprises a radiopaque marker band disposed on the tubular wall adjacent the distal end, the radiopaque marker band being disposed between the tubular liner and the tubular wall.

13. The apparatus according to claim 1, wherein the outer sheath further comprises a radiopaque marker band disposed on the tubular wall adjacent the distal end, the radiopaque marker band comprising at least one of platinum, gold, silver, palladium, tungsten, niobium, cobalt, and copper.

14. A delivery assembly for introducing an implant for capturing emboli in a body vessel, the assembly comprising:
an outer sheath comprising a tubular wall defining a lumen formed therethrough and having a proximal end extending to a distal end, the tubular wall having an inner surface, a tubular liner disposed along the inner surface, the tubular liner comprising at least one of polyimide, PEEK, and PVDF;
an inner catheter slidably disposed within the lumen of the outer sheath, the inner catheter having a proximal portion extending to a distal portion, the inner catheter being configured to longitudinally move relative to the outer sheath;
a vena cava filter having a proximal segment extending to a distal segment, the distal segment including a retrieval hook, a plurality of diverging spring-biased centering legs and a plurality of diverging spring-biased anchoring legs being attached together at the distal segment, the anchoring legs having proximal free ends including anchoring hooks for engaging the body vessel, the centering legs providing a force to center the implant in the body vessel, and
the inner catheter being configured to engage and deliver the vena cava filter through the distal end of the outer sheath in the body vessel such that the retrieval hook of the distal segment of the filter releasably engages the distal portion of the inner catheter.

15. The apparatus according to claim 14, wherein reinforcing members are disposed within the tubular wall.

16. The apparatus according to claim 15, wherein the reinforcing members are made of stainless steel or high strength plastic or a mixture thereof.

17. The apparatus according to claim 16, wherein the high strength plastic includes aramid fibers.

18. The apparatus according to claim 14, wherein the outer sheath further comprises a radiopaque marker band disposed about the inner surface adjacent the distal end, the radiopaque marker band being disposed between the inner surface and the tubular liner.

19. The apparatus according to claim 14, wherein the outer sheath further comprises a radiopaque marker band disposed about the inner surface adjacent the distal end, the radiopaque marker band comprising at least one of platinum, gold, silver, palladium, tungsten, niobium, cobalt, and copper.

20. A method of introducing an implant into a body vessel, the method comprising:
providing a delivery apparatus within the body vessel, the delivery apparatus comprising an outer sheath including a tubular wall defining a lumen formed therethrough, the tubular wall having an inner surface and a tubular liner being disposed along the inner surface, the tubular liner comprising at least one of polyimide, PEEK, and PVDF, a radiopaque marker band being disposed near the distal end of the outer sheath, an inner catheter being slidably disposed within the lumen of the outer sheath;
inserting the implant within the lumen such that a distal retrieval hook of the implant is releasably coupled to a distal portion of the inner catheter and the implant is adjacent a distal end of the delivery apparatus;
positioning the distal end of the delivery apparatus adjacent a desired location in the body vessel;
retracting the outer sheath proximally from the implant; and
releasing the implant into the body vessel.
